(19) United States
(12) Plant Patent Application Publication
(10) Pub. No.: US 2003/0033647 P1
(43) Pub. Date: Feb. 13, 2003

(54) Weigela plant named 'Pink Poppet'

(76) Inventors: Ing Vojtech Benetka, Chodov (CZ); Jaroslava Dvorakova, Cestlice (CZ)

Correspondence Address: MARK P. BOURGEOIS, P.O. BOX 95, OSCEOLA, IN, 46561, US

(21) Appl. No.: 09/925,696

(22) Filed: Aug. 9, 2001

(51) Int. Cl. A01H 5/00

(52) U.S. Cl. PLT/226

(57) ABSTRACT

A new cultivar of Weigela named ‘Pink Poppet’ that is characterized by a compact dwarf habit, winter hardiness and light pink flowers that bloom in early spring. In combination these traits set ‘Pink Poppet’ apart from all other existing varieties of Weigela.

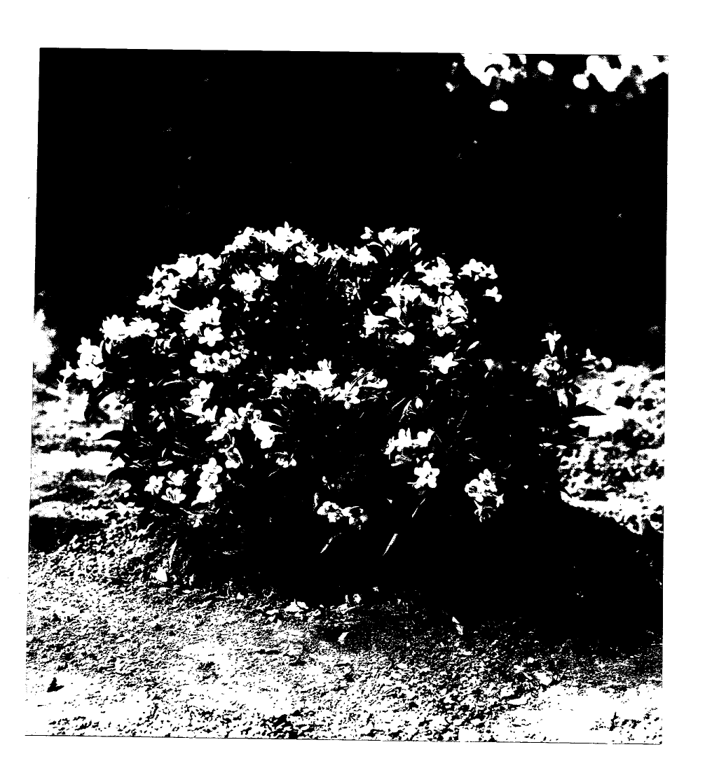

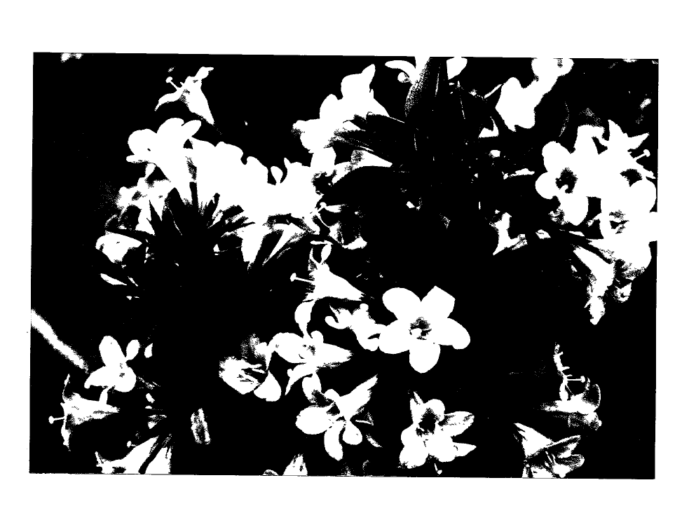

### BACKGROUND OF THE INVENTION

The present invention relates to a new and distinct cultivar that is a low growing deciduous shrub with a compact habit. The new cultivar is known botanically as a Weigela and will be referred to hereinafter by the cultivar name ‘Pink Poppet’.

‘Pink Poppet’ is part of a breeding program and was bred by the inventors at the Research Institute of Ornamental Gardening Pruhonice, Czech Republic. The Research Institute is a non-profit organization. The goal of the breeding program is to produce a series of dwarf and compact shrubs that exhibit various flower colors and are usable out of doors. At the Research Institute, crossing the seed parent Weigela florida ‘Venusta’ (not patented) with the pollen parent Weigela hybrida ‘Eva Rathke’ (not patented). Four seedlings of F1 generation were produced by this interspecific hybridization. Harvested seeds of the F2 generation were planted in 1984 and a total of 189 seedlings were produced. The F2 generation was cultivated for seven years. The seven generations of vegetative propagadion were conducted to ensure uniformity and stability. ‘Pink Poppet’ was selected in 1991 based on habit, hardiness, flower color, abundance of flowers and number of flushes. The traits that make ‘Pink Poppet’ unique are dwarf growth habit and density, light pink flowers, early flowering, winter hardiness and its strong competition in controlling weed growth. The closest comparison plants are the parents. ‘Pink Poppet’ is distinguishable from both parent plants by its compact size, smaller leaf, shorter internodes, smaller stems and flower color.

In 1991 ‘Pink Poppet’ was first asexually propagated by the inventors at the Research Institute of Ornamental Gardening Pruhonice, Czech Republic using the method of softwood cuttings. Since that time, under careful observation, the distinguishing characteristics have been determined stable and uniform in successive generations.

### SUMMARY OF THE INVENTION

The following represent the distinguishing characteristics of the new Weigela cultivar ‘Pink Poppet’. In combination these traits set ‘Pink Poppet’ apart from all other existing varieties of Weigela known to the inventors. ‘Pink Poppet’ has not been tested under all possible conditions and phenotypic differences may be observed with variations in environmental, climatic, and cultural conditions, however, without any variance in genotype.

1. Weigela ‘Pink Poppet’ exhibits two flushes of light pink flowers in spring.

2. Weigela ‘Pink Poppet’ exhibits a compact dwarf growth habit.

3. Weigela ‘Pink Poppet’ is hardy to USDA Zone 5.

4. Weigela ‘Pink Poppet’ is 0.6 meter in height and 1 meter in width at maturity.

5. Weigela ‘Pink Poppet’ is strongly competitive in controlling growth of weeds.

### BRIEF DESCRIPTION OF THE DRAWINGS

The accompanying color photographs illustrate the distinguishing traits of Weigela ‘Pink Poppet’. The plants in the photographs were grown out of doors at the Research Institute in Pruhonice, Czech Republic. The photograph on sheet 1 illustrates the entire plant in bloom from a side perspective. The photograph on sheet 2 is a close-up view of the flowers. The photographs were taken using conventional techniques and although colors may appear different from actual colors due to light reflectance, they are as accurate as possible by conventional photography.

### BOTANICAL DESCRIPTION OF THE PLANT

The following is a detailed description of the Weigela cultivar named ‘Pink Poppet’. Data was collected in Arroyo Grande, Calif. Color determinations are in accordance with The Royal Horticultural Society Colour Chart except where general color terms of ordinary dictionary significance are used. The growing requirements are similar to the species and there are no disease problems known to the inventors.

Botanical classification: Weigela ‘Pink Poppet’.

Use: Ornamental landscape shrub that offers strong competition in controlling the growth of weeds.

Parentage: ‘Pink Poppet’ is a hybrid that resulted from the induced hybridization of the foi owing plants:

Female.—Weigela florida ‘Venusta’ (not patented).

Male.—Weigela hybrida ‘Eva Ratlike’ (not patented).

Vigor: Weak.

Growtlh habit. Erect to prostrate and spreading.

Size: Dwarf.

Height: 0.6 meter in height.

Width: 1 meter in width.

Hardiness: USDA Zone 5.

Propagaion: Softwood cuttings.

Root system: Fibrous.

Light: Plant in full sun or dappled shade.

Soil: Plant in moist but well drained soil.

Type: Shrub.

Seasonal interest: Two flushes of flowers in spring.

Stem:

Branching habit.—Upright branching.

Stem diameter.—3.4 mm. in diameter

Internode length.—35.8 mm. between nodes.

Shape.—Tetrahedal.

Surface.—Pubescent.

Branch color.—Light brown.

Foliage:

Type.—Deciduous.

Leaf arrangement.—Opposite.

Leaf division.—Simple.

Leaf margin.—Serrate.

Leaf surface (adaxial and abaxial).—Pubescent.

Leaf shape.—Elliptic to obovate.

Leaf apex.—Caudate.

Leaf attachment.—Petiolate.

Stipules.—Absent.

Petiole dimensions.—5 mm. in length and 1 mm. in width.

Leaf length.—78.2 mm. in length.

Leaf width.—31.5 mm. in width.

Leaf color (adaxial surface).—147A.

Leaf color (abaxial surface).—147A.

Flower:

Inflorescence.—Axillary corymb.

Flower form.—Solitary.

Shape of flower.—Funnelform.

Dimensions of flower.—33 mm. in height and 29.5 mm. in diameter.

Aspect.—Facing outward and upward.

Color of flower petal (outside).—70B, C.

Color of flower petal (inside).—75C.

Corolla surface (outer).—Puberulent.

Petals.—Five in number.

Petal surface.—Puberulent.

Unfused or fused.—Petals are basally fused.

Bud color.—71B,C.

Lobes.—Five imbricate lobes when in bud.

Number of sepals in calyx.—Five sepals in the calyx.

Length of sepals.—8.1 mm. in length.

Blooming period.—May to June.

Reprodvction organs:

Stamens.—Five in number.

Color of filaments.—Green-white.

Anther color.—Cream-white.

Stigma color.—White.

Style color.—Green-white.

Style.—Exerted.

Seed: Seed production has not been observed.

1. A new and distinct variety of weigela plant named ‘pink poppet’ as described and illustrated.
